(19) United States
(12) Patent Application Publication
(10) Pub. No.: US 2025/0127584 A1
(43) Pub. Date: Apr. 24, 2025

(54) DEVICE FOR COVERING AN AREA OF THE BODY OF A PATIENT

(71) Applicant: Cook Medical Technologies LLC, Bloomington, IN (US)

(72) Inventors: Juan Sebastián Sánchez López, Magdeburg (DE); Sinja Lagotzki, Magdeburg (DE)

(73) Assignee: Cook Medical Technologies LLC, Bloomington, IN (US)

(21) Appl. No.: 18/698,986

(22) PCT Filed: Oct. 5, 2022
(86) PCT No.: PCT/EP2022/077684

§ 371 Date: Dec. 2, 2024

(30) Foreign Application Priority Data
Oct. 6, 2021 (DE) 10 2021 125 935.5

(51) Int. Cl. A61B 46/20; A61B 90/00

(52) U.S. Cl. CPC A61B 46/20

(57) ABSTRACT

The invention relates to a device for covering at least one area of the body of a patient during a medical procedure, wherein the device has a flat, thin, sterile drape and a marking grid connected to the drape, which has markings for determining a position in CT and/or MRI scans, wherein the marking grid is attached to the drape via a detachable connection so that it can be removed from the drape during the course of the medical procedure without the need to remove the drape from the patient.

The invention relates to a device for covering at least one area of the body of a patient during a medical procedure, in particular a surgical procedure, by means of a flat, thin, sterile drape which comprises an integrated marking grid which has markings for determining the position in CT and/or MRI scans. Drape cloths are generally known in the medical field and are, for example, referred to as surgical drapes, OP drapes or drapes. In the following, the term “surgical drape” or just “drape” is used for such drapes.

Surgical drapes are used in the operating room (OR) to protect patients, clinical staff, and equipment. Such drapes are often designed as disposable items. Important features are the barrier protection effectiveness, the resistance to ignition, and the durability. Surgical drapes are used to form a physical barrier around the patient to protect the surgical field from contamination. Drapes are placed in the surgical field around an incision site to cover the patient and absorb fluids. The drape is intended to prevent contamination of the surgical wound and the surgical instruments used.

As mentioned, such surgical drapes are known. The invention is based on the task of providing an improved device for covering at least one area of the body of a patient during a medical procedure, which device can be used more universally and simplifies the procedure on the patient.

This task is solved by a device for covering at least one area of the body of a patient during a medical procedure, wherein the device comprises a flat, thin, sterile drape and a marking grid connected to the drape, which has markings for determining the position in CT and/or MRI scans, wherein at least a part of the marking grid is attached to the drape via a detachable connection so that it can be removed from the drape during the course of the medical procedure without the need to remove the drape from the patient. CT means computed tomography and MRI means magnetic resonance imaging. The entire marking grid or at least a part of the marking grid can thus be removed from the drape during the course of the medical procedure by loosening the detachable connection, while the drape remains on the patient. The invention has the advantage that procedures on the patient that require a marking grid for CT and/or MRI scans can be considerably simplified and accordingly accelerated. In this way, the burden on both the patient and the clinical staff is reduced.

The device according to the invention is thus provided to the user in its complete state, which is to say, with the drape and the marking grid attached to the drape.

The detachable connection can, for example, be a material and/or a form-fit connection. In the case of a material connection, an adhesive connection with a comparatively easily detachable adhesive can, for example, be used, in a manner similar to an adhesive for sticky notes. A form-fit detachable connection can be realized, for example, by using hook and loop fastener tape. Another embodiment of a suitable form-fit connection is a zipper or a zip lock connection.

The drape comprises at least one cut-out, at least when at least part of the marking grid has been removed from the drape.

By way of example, the marking grid can consist of the material of the drape or of another material. In an advantageous embodiment, the marking grid can be applied to the material of the drape, for example, by printing on corresponding grid-like markings. The detachable connection between the marking grid and the surrounding drape can be realized, for example, by perforation lines or similar separation lines. In this case, the at least one cut-out in the drape is only created by removing the marking grid. In other embodiments, which will be described below, the drape can have the at least one cut-out from the outset. In the initial state of the device, the at least one cut-out is then covered by the marking grid.

In such a procedure on the patient, for example, a biopsy, in the prior art, at least the following ten steps are required:
1) Placement of a (separate) marking grid on the patient
2) Performance of the CT and/or MRI scan on the patient for the planning of the procedure on the patient
3) Marking of the desired entry point of a surgical instrument on the patient using the marking grid
4) Removal of the marking grid from the patient
5) Cleansing and sterilization of the surrounding area around the marked entry point
6) Placement of a conventional surgical drape on the body of the patient
7) Performance of a new CT and/or MRI scan on the patient
8) Verification of the marked entry point based on the new CT and/or MRI scan
9) Introduction of an anesthetic agent in the area of the marked entry point
10) Performance of the medical procedure at the marked entry point

This process can now be simplified as follows using the device according to the invention:
1) Cleansing and sterilization of the surrounding area around the desired entry point
2) Placement on the patient of the device according to the invention with the drape and the marking grid covering the at least one cut-out and attached to the drape
3) Performance of the CT and/or MRI scan on the patient for the planning of the procedure on the patient
4) Marking the desired entry point of a surgical instrument on the patient, wherein the local anesthetic can be introduced at the same time
5) Removal of the marking grid from the drape
6) Performance of the medical procedure at the marked entry point

In this respect, the present invention also includes methods for prepping the patient for a medical procedure. The method is characterized, for example, in that the desired entry point is marked on the patient after the patient has been covered with the drape. In a further advantageous embodiment, the method provides for marking the desired entry point by introducing the local anesthetic agent at the desired entry point. In a further advantageous embodiment of the method, it is provided that the marking grid is removed from the patient already covered with the drape. According to a further advantageous embodiment of the method, the CT and/or MRI scan is performed on the patient who has already been covered with the drape.

The marking grid can have one or a plurality of markers that are visible in a CT scan or an MRI scan. The marking grid can, in particular, be attached to the drape by means of a connection that can be detached without tools, so that it can be removed from the drape without additional tools such as a knife, scissors or the like. If the marking grid is removed from the drape, then the cut-out is exposed and allows for a direct access to the patient.

According to an advantageous embodiment of the invention, it is provided that the device is packaged in sterile packaging with the drape and the marking grid attached to the drape and covering the at least one cut-out, if present. The device according to the invention in its complete configuration can thus be provided as a sterile product in the sterile packaging. The user then only needs to unpack the device from the packaging and it can be used directly on the patient.

According to an advantageous embodiment of the invention, it is provided that the marking grid has an outer frame and a grid area surrounded by the outer frame, which grid area has the markings for determining the position in CT and/or MRI scans. This has the advantage that the marking grid can be connected to the drape by means of a defined mechanical interface, namely the outer frame. By way of example, it might only be the outer frame that has a direct connection to the drape. It can also be that the marking grid or alternatively the grid area has at least a partial connection to the drape, for example, at the edge. The outer frame can be configured as an element permanently attached to the drape, which remains attached even after the marking grid has been removed from the drape and accordingly also remains there during the medical procedure on the patient. Correspondingly, the detachable connection, by means of which at least part of the marking grid is attached to the drape, is formed between the outer frame and the grid area.

In an advantageous embodiment of the invention, the outer frame is permanently attached to the drape, in particular by means of a non-detachable connection, and the grid area is attached to the outer frame by means of the detachable connection, so that over the course of a medical procedure the grid area can be removed from the outer frame and thus from the drape without tools by detaching the detachable connection, without the drape needing to be removed from the patient, wherein the outer frame remains attached to the drape.

According to an advantageous embodiment of the invention, it is provided that the grid area is connected to the outer frame in a material and/or form-fitting manner. For the connection between the grid area and the outer frame, the previously explained possibilities of material and/or form-fitting connections can be used. In addition, a material connection between the grid area and the outer frame can be realized by one or a plurality of material bridges which extend from the grid area to the outer frame, for example, wherein the grid area is formed integrally with the outer frame. In this case, it is advantageous if the grid area, at least in the area of its outer edge, is made of the same material as the outer frame. In principle, it is, however, possible for the grid area and the outer frame to be made of different materials.

According to an advantageous embodiment of the invention, it is provided that the material-fitting connection between the grid area and the outer frame has one or a plurality of predetermined breaking points. This has the advantage that the grid area can be separated from the outer frame by the user in a simple and reliable manner, in particular without tools. By way of example, when compared to the material thickness of the grid area and the outer frame, the predetermined breaking points can be configured as a relatively thin material bridge or a plurality of material bridges.

According to an advantageous embodiment of the invention, it is provided that the outer frame is attached to the drape by means of a non-detachable connection. A non-detachable connection is considered to be a connection that cannot be detached without tools. By way of example, the outer frame can be attached to the drape by a permanent adhesive bond. The outer frame can also be welded to the drape.

According to an advantageous embodiment of the invention, it is provided that the grid area is connected to the outer frame by means of a peelable connecting element, wherein the peelable connecting element can be peeled away from the grid area and the outer frame essentially without leaving any residue and without tools. This too makes it possible to remove the grid area from the drape easily and without tools. The peelable connecting element can, for example, be designed similarly to a pull-off tab on a package.

In the original state of the device, the peelable connecting element can, for example, be connected to the grid area and/or to the outer frame by one or more material bridges or be formed integrally therewith.

According to an advantageous embodiment of the invention, it is provided that the grid area consists entirely or at least partially of a material that is visible in a CT scan or an MRI scan. This has the advantage that the grid area or at least a part thereof is directly visible in the images generated during the CT or MRI scan, which is helpful for planning and performing the medical procedure. By way of example, certain marking lines of the grid area may consist entirely or at least partially of a material that is visible in a CT scan or an MRI scan.

According to an advantageous embodiment of the invention, it is provided that a handle element is arranged on the marking grid, which is arranged for manually gripping the marking grid for removal from the drape, wherein the handle element is not or is less firmly attached to the drape than the parts of the marking grid attached to the drape by means of the detachable connection. This has the advantage that the marking grid or at least the grid area can easily be gripped manually by the user when it is to be removed. The handle element can, for example, be configured as a grip tab that protrudes beyond the outer contour of the marking grid or grid area. When the marking grid is configured with the grid area and the outer frame, the handle element can, for example, be attached to the grid area but not to the outer frame.

According to an advantageous embodiment of the invention, it is provided that the handle element is arranged in a manner overlapping the outer frame. This has the advantage that the handle element can readily be gripped by the user, but does not protrude too far outwards.

According to an advantageous embodiment of the invention, it is provided that the device has at least one self-adhesive area at least in the area of the at least one cut-out on the side facing the patient. This has the advantage that the device and in particular the drape can be reliably positioned on the patient and the risk of unintentional slippage of the cut-out area can be minimized. When the device is in as-delivered condition, the self-adhesive area can be covered by a peelable protective cover, for example, a cover film. This protective cover can then be easily peeled off by hospital staff before attaching the device to the patient. The self-adhesive area can, for example, be located on the side of the outer frame of the marking grid facing the patient.

According to an advantageous embodiment of the invention, it is provided that the cut-out and/or the outer edge of the grid area has a rectangular or square contour with rounded corners, a round contour, an oval contour or the contour of any polygon. In this way, for example, a marking grid with a conventional shape can be provided, which can be removed from the outer frame particularly easily and reliably due to its rounded corners.

According to an advantageous embodiment of the invention, it is provided that the cut-out is completely surrounded on the circumferential side by the material of the drape and/or the outer frame.

The marking grid or alternatively the grid area can have any desired design (shape), for example, with grid bars that are orthogonal to one another or in the manner of a spider web, which is to say, with grid bars extending radially from a center, which are intersected by several ring-shaped grid bars encircling the center.

The invention is explained in more detail below with reference to embodiment examples using drawings.

Wherein:

FIG. 1 shows a device for covering an area of the body of a patient in perspective view,

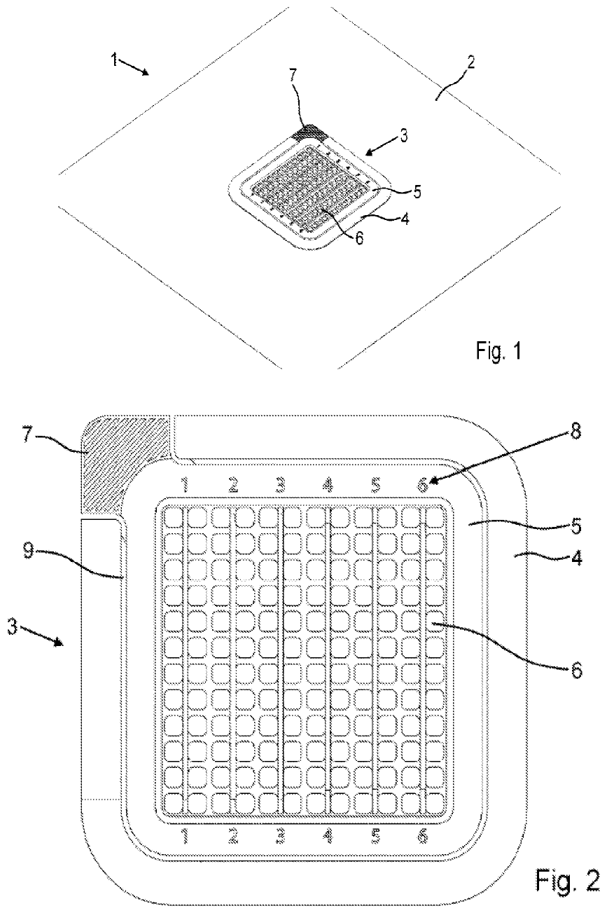

FIG. 2 shows a top view of a marking grid,

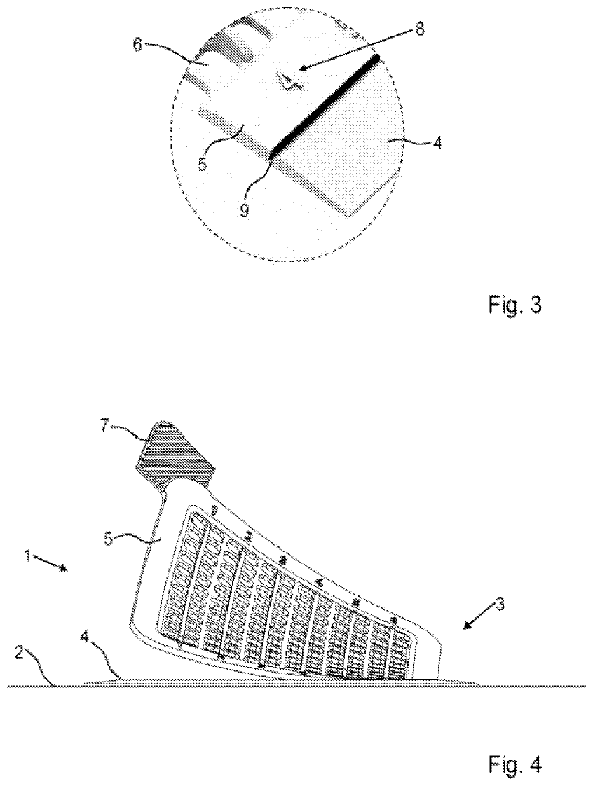

FIG. 3 shows an enlarged section of a part of the marking grid according to FIG. 2 in perspective view,

FIG. 4 shows the device according to FIG. 1 with a partially peeled off grid area,

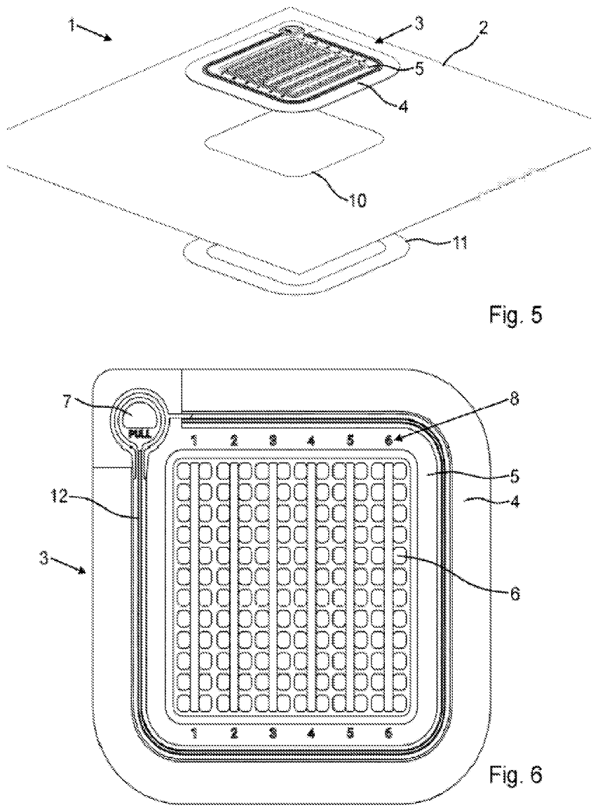

FIG. 5 shows a further embodiment of a device for covering an area of the body of a patient in an exploded perspective view,

FIG. 6 shows the marking grid of the device according to FIG. 5a in plan view,

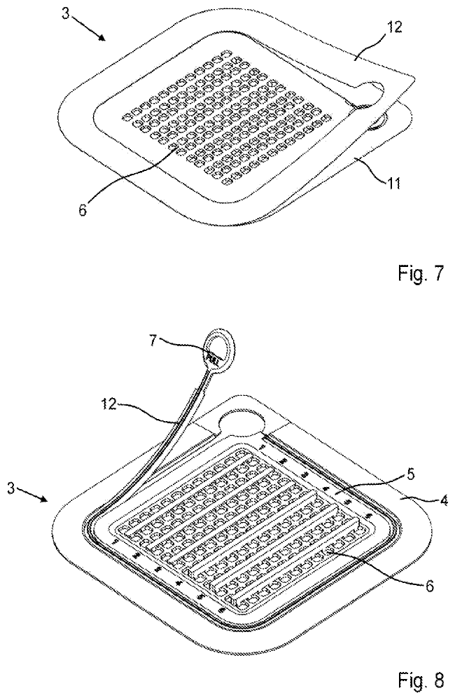

FIG. 7 shows a view of the device on the side of the drape facing the patient,

FIG. 8 shows the marking grid on the side facing away from the patient during the peeling off process.

The device 1 depicted in FIG. 1 for covering at least one area of body of a patient during a medical procedure comprises a flat, thin, sterile drape 2 with a cut-out which, in the embodiment shown, is already covered with a marking grid 3. The marking grid 3 comprises markings 6 for determining the position in CT and/or MRI scans, for example, a matrix-like grid.

The marking grid 3 has an outer frame 4 and a grid area 5 surrounded by the outer frame 4, which grid area contains the markings 6. The outer frame 4 is permanently connected to the drape 2. The grid area 5 is attached to the outer frame 4 and thus to the drape 2 by means of a detachable connection. The grid area 5 is the part of the marking grid 3 that can be removed from the drape 2 during the course of the medical procedure without the drape 2 needing to be removed from the patient.

FIG. 2 shows an enlarged view of the marking grid 3 with the elements elucidated. It can be seen that additional information for orientation may be present in the grid area 5, for example, a scale 8 provided with numbers. In the figures, the scale 8 is only shown in one coordinate direction. Such a scale 8 can of course also be present in a further coordinate direction. In the embodiment shown, the grid area 5 is connected in one piece to the outer frame 4 by means of a thin material bridge 9. The material bridge 9 is shaped in such a way that it contains at least one predetermined breaking point through which the grid area 5 can be easily detached from the outer frame 4 by breaking open the predetermined breaking point without needing to remove the drape 2 from the patient or without the drape 2 significantly changing its position when the grid area 5 is removed.

In order to be able to easily grip the grid area 5 manually during the release process from the drape 2, a manual handle element 7 can be arranged in a corner area, for example, in the form of a grip tab that is firmly connected to the grid area 5. In contrast, the handle element 7 is not connected to the outer frame 4 and/or the drape 2, or is at least less firmly connected than the detachable connection between the grid area 5 and the outer frame 4.

In a further enlarged view, FIG. 3 shows a possible realization of the material bridge 9, for example, in the form of a wedge-shaped tapering material bridge between the grid area 5 and the outer frame 4.

FIG. 4 shows the device 1 in side view, wherein the marking grid 3 or alternatively its grid area 5 has already been partially removed from the drape 2.

A further advantageous embodiment of the device according to the invention is elucidated on the basis of FIG. 5 through FIG. 8. FIG. 5 shows a similar perspective view to FIG. 1, wherein the elements to be attached to the drape 2 are shown at a distance from it for better illustration, in the sense of an exploded view. When the device 1 is in as-delivered condition, these elements are in turn attached to the drape 2, as shown in FIG. 1.

One can recognize a cut-out 10 in the drape 2 in FIG. 5, which cut-out is covered by the marking grid 3. The marking grid 3 is attached to the side of the drape 2 facing away from the patient. In addition, an adhesive element 11 can be attached to the side of the drape 2 facing the patient, by means of which a self-adhesive area can be formed on the drape 2.

FIG. 6 shows an enlarged top view of the marking grid 3. In contrast to the embodiment of FIG. 1 through FIG. 4, in this case a detachable connecting element 12 is provided as a detachable connection between the outer frame 4 and the grid area 5, which detachable connecting element is coupled to the handle element 7. The peelable connecting element 12 extends over all four circumferential sides of the grid area 5. The detachable connecting element 12 is respectively coupled on the one hand to the outer frame 4 by means of one or a plurality of material bridges and on the other hand to the grid area 5 by means of one or a plurality of material bridges. By pulling on the handle element 7, the peelable connecting element 12 can be separated from the outer frame 4 and the grid area 5 in the manner of a pull cord, wherein by continuously pulling on the handle element 7, the detachable connecting element 12 is released circumferentially from the grid area 6 and the outer frame 4, similar to opening a zipper. After pulling off the peelable connecting element 12, the grid area 5 can be removed with minimal effort and without moving the drape 2.

FIG. 7 shows a view of the device 1 on the side of the drape 2 facing the patient. Prior to attachment of the device 1 to the patient, a protective film must first be removed from the adhesive element 11 so that the self-adhesive area is exposed and can be attached to the patient.

FIG. 8 shows the marking grid 3, once again on the side of the drape 2 facing away from the patient during the peeling off process of the peelable

1-14. (canceled)

15. A device for covering at least one area of the body of a patient during a medical procedure, wherein the device comprises a flat, thin, sterile drape and a marking grid connected to the drape, which has markings for determining a position in CT and/or MRI scans, wherein at least a part of the marking grid is attached to the drape via a detachable connection so that it can be removed from the drape during the course of the medical procedure without the need to remove the drape from the patient.

16. The device according to claim 15, characterized in that the drape comprises at least one cut-out at least when at least a part of the marking grid has been removed from the drape.

17. The device according to claim 15, characterized in that the device is packaged in a sterile package with the drape and the marking grid attached to the drape.

18. The device according to claim 15, characterized in that the marking grid has an outer frame and a grid area surrounded by the outer frame, which grid area has the markings for determining the position in CT and/or MRI scans.

19. The device according to claim 18, characterized in that the grid area is connected to the outer frame in a material and/or form-fitting manner.

20. The device according to claim 10, characterized in that the material-fitting connection between the grid area and the outer frame has one or a plurality of predetermined breaking points.

21. The device according to claim 18, characterized in that the outer frame is attached to the drape by a non-detachable connection.

22. The device according to claim 18, characterized in that the grid area is connected to the outer frame by a peelable connecting element, wherein the peelable connecting element can be peeled away from the grid area and the outer frame essentially without leaving any residue and without tools.

23. The device according to claim 18, characterized in that the grid area comprises a material that is visible in a CT scan or an MRI scan.

24. The device according to claim 15, characterized in that a handle element is arranged on the marking grid, which is arranged for manually gripping the marking grid for removal from the drape, wherein the handle element is not or is less firmly attached to the drape than parts of the marking grid attached to the drape by the detachable connection.

25. The device according to claim 24, characterized in that the handle element is arranged in a manner overlapping the outer frame.

26. The device according to claim 15, characterized in that the device has at least one self-adhesive region at least in the region of the at least one cut-out on a side of the device configured to face the patient.

27. The device according to claim 18, characterized in that the cut-out and/or an outer edge of the grid area has a rectangular or square contour with rounded corners, a round contour, an oval contour or the contour of any polygon.

28. The device according to claim 15, characterized in that the cut-out is completely circumferentially surrounded by a material of the drape and/or the outer frame.

29. The device according to claim 18, wherein:
the marking grid comprises an outer frame, and the grid area is surrounded by the outer frame;
the grid area is connected to the outer frame and the outer frame is connected to the drape;
the connection between the grid area and the outer frame has one or a plurality of predetermined breaking points; and
the drape comprises at least one cut-out exposed when the grid area of the marking grid has been detached from the drape.

30. The device according to claim 29, characterized in that the grid area is connected to the outer frame by a peelable connecting element, wherein the peelable connecting element can be peeled away from the grid area and the outer frame to remove the grid area from the drape.

31. The device according to claim 30, wherein the peelable connecting element is formed integrally with the grid area and the outer frame.

32. The device according to claim 30, wherein the peelable connecting element is coupled to a handle element.

33. A device for covering at least one area of the body of a patient during a medical procedure, comprising:
a sterile drape having an opening therethrough;
a marking grid connected to the sterile drape and covering the opening;
wherein the marking grid includes an outer frame and a grid area surrounded by the outer frame, with the grid area being connected to the outer frame by a detachable connection including one or a plurality of predetermined breaking points, and with the outer frame being connected to the sterile drape;
wherein the grid area has markings that are visible in a CT scan or an MRI scan; and
wherein separation of the grid area from the outer frame via the detachable connection exposes the opening.

34. The device according to claim 33, wherein the detachable connection comprises a peelable connecting element connecting the grid area to the outer frame, wherein the peelable connecting element is configured to be peeled away from the grid area and the outer frame to separate the grid area from the drape so as to expose the opening.
